(19) United States
(12) Patent Application Publication
(10) Pub. No.: US 2024/0216458 A1
(43) Pub. Date: Jul. 4, 2024

(54) Composition for the prevention, improvement or treatment of allergic diseases comprising an extract of Cassia mimosoides L. as an active ingredient

(71) Applicant: KOREA INSTITUTE OF SCIENCE AND TECHNOLOGY, Seoul (KR)

(72) Inventors: Myung Suk KIM, Gangneung-si (KR); Sang Hoon JUNG, Gangneung-si (KR); Wook Bin LEE, Gangneung-si (KR); Tam Thi LE, Gangneung-si (KR); Tae Kyeom KANG, Gangneung-si (KR)

(73) Assignee: KOREA INSTITUTE OF SCIENCE AND TECHNOLOGY, Seoul (KR)

(21) Appl. No.: 18/540,186

(22) Filed: Dec. 14, 2023

(30) Foreign Application Priority Data
Dec. 15, 2022 (KR) 10-2022-0176051

(51) Int. Cl. A61K 36/482; A61K 31/05; A61K 31/085; A61K 31/122; A61K 31/192; A61K 31/216; A61K 31/353; A61K 31/7048; A61P 37/08

(52) U.S. Cl. CPC A61K 36/482; A61K 31/05; A61K 31/085; A61K 31/122; A61K 31/192; A61K 31/216; A61K 31/353; A61K 31/7048; A61P 37/08; A61K 2236/333

(57) ABSTRACT

The present invention relates to a composition for the prevention, improvement or treatment of allergic diseases containing a Cassia mimosoides L. extract as an active ingredient, and more specifically, to a composition for the prevention, improvement or treatment of allergic diseases containing an ethanol extract of Cassia mimosoides L. as an active ingredient. In the present invention, the effect of Cassia mimosoides L. extract in treating allergic respiratory diseases was confirmed in an animal model of allergic asthma, and the anti-allergic effect of the compounds isolated from an ethanol extract of Cassia mimosoides L. was confirmed. Therefore, Cassia mimosoides L. extract of the present invention can be useful as a composition for preventing, improving, or treating allergic respiratory diseases, particularly allergic asthma or allergic bronchitis.

### CROSS-REFERENCE TO RELATED APPLICATIONS

This application claims the benefit under 35 USC 119(a) of Korean Patent Application No. 10-2022-0176051 filed on Dec. 15, 2022, in the Korean Intellectual Property Office, the entire disclosure of which is incorporated herein by reference for all purposes.

### TECHNICAL FIELD

The present invention relates to a composition for the prevention, improvement or treatment of allergic diseases containing an extract of Cassia mimosoides L. as an active ingredient, and more specifically, to a composition for the prevention, improvement or treatment of allergic diseases containing an ethanol extract of Cassia mimosoides L. or fraction(s) thereof as an active ingredient.

### BACKGROUND ART

Allergic diseases include anaphylaxis, allergic rhinitis, asthma, atopy, and urticaria. Allergic diseases such as the above occur in up to 20% of the population in many countries around the world, and the prevalence rate is increasing.

Allergic diseases are mostly caused by mediators (mainly histamine, leukotrienes, prostaglandins, TNFα, cytokines, etc.) released from tissue mast cells and blood basophils and eosinophils activated by antigen-antibody reactions.

In particular, allergic asthma is an immune disease of the respiratory mucosa that causes airway inflammation, mucus hypersecretion, and airway hyperreactivity. Airway inflammation is caused by inflammatory cells such as T cells, B cells, mast cells, neutrophils, and eosinophils, and chemical mediators such as cytokines and chemokines, and is characterized by airway hyperresponsiveness, lung inflammation, and increased secretion of IgE in the serum (Herrick C A and Bottomly K., Nat Rev Immunol., 3(5):405-412, 2003).

Anti-histamines, leukotriene inhibitors, steroids, etc. have been used to treat allergic diseases, and recently, protein- and peptide-based allergy treatments that block histamine and interleukin receptors are being developed. However, since the use of allergy treatment drugs is limited to symptom relief, the development of more fundamental treatment drugs is urgently needed (Kips J C, et. al., Am J Respir Crit Care Med., 167(12):1655-1659, 2003).

Meanwhile, Cassia mimosoides L. is an annual herb of the dicotyledonous Rosaceae legume family. The dried whole plant of Cassia mimosoides L. is called mountain lentil, and it is effective against jaundice caused by moist heat and vomit caused by food poisoning. The whole plant and seeds contain aloeemodin, and the seeds contain myristic acid, palmitic acid, and β-sitosterol (Components of medicinal herbs and usage thereof, Science Encyclopedia Publishing, Ilwolseogak, 1991). In the prior art, it has been known about the prevention and treatment effects of Cassia mimosoides L. on ischemic diseases and the anti-skin aging and anti-wrinkle effects (Korean Patent Publication No. 10-2006-0080131; Korean Patent Publication No. 10-2008-0036302).

### DISCLOSURE

### Technical Problem

Accordingly, in the present invention, the present inventors made diligent efforts to select substances that are effective in treating allergic diseases, especially allergic respiratory diseases. As a result, the present inventors not only confirmed therapeutic effect of Cassia mimosoides L. extract in allergic respiratory diseases in animal models of allergic asthma, but also confirmed anti-allergy efficacy of the compounds isolated from an ethanol extract of Cassia mimosoides L., and the present invention was completed.

Accordingly, the purpose of the present invention is to provide a pharmaceutical composition for preventing or treating allergic respiratory diseases containing a Cassia mimosoides L. extract or fraction(s) thereof.

Another object of the present invention is to provide a health functional food composition for preventing or improving allergic respiratory diseases containing Cassia mimosoides L. extract or fraction(s) thereof.

### Technical Solution

To achieve the above-mentioned purpose, the present invention provides a pharmaceutical composition for the prevention or treatment of allergic respiratory diseases comprising an extract of Cassia mimosoides L. (also referred to “Cassia mimosoides L. extract”) or fraction(s) thereof as an active ingredient.

In addition, the present invention provides a health functional food composition for preventing or improving allergic respiratory diseases containing Cassia mimosoides L. extract or fraction(s) thereof as an active ingredient.

In a preferred embodiment of the present invention, Cassia mimosoides L. extract can be extracted using 50% (v/v) to 100% (v/v) ethanol as a solvent.

In another preferred embodiment of the present invention, the fraction may be an ethyl acetate fraction of 60% (v/v) to 80% (v/v) ethanol extract of Cassia mimosoides L. (also referred to “Cassia mimosoides L. ethanol extract”).

In another preferred embodiment of the present invention, the allergic respiratory disease may be allergic asthma, allergic bronchitis, or allergic rhinitis.

In another preferred embodiment of the present invention, Cassia mimosoides L. extract can inhibit the secretion of beta-hexosaminidase, allergens present in the granules of mast cells, or inhibit the production of histamine, TNF-α, interleukin-4 (IL-4), or prostaglandin E2 secreted by mast cells.

In another preferred embodiment of the present invention, Cassia mimosoides L. extract may inhibit or ameliorate allergic symptoms by reducing the number of eosinophils or inflammatory cells in the bronchi; reducing eosinophil infiltration; or reducing IgE in the serum.

In another preferred embodiment of the present invention, Cassia mimosoides L. extract may contain any one or more compounds selected from the group consisting of:
Drimiopsin H represented by the following formula 3;
Altechromone A represented by the following formula 4;
Ethyl caffeate represented by the following formula 5;
Isorhapontigenin represented by the following formula 7;
KA-1 compound [(2S)-3′,4′,7-trihydroxyflavan-(4β->8)-catechin] represented by the following formula 13;
(2S)-4′,7-dihydroxyflavan-(4β->8)-catechin represented by the following formula 14;
KA-2 compound [(2S)-3′,4′,7-trihydroxyflavan-(4α->8)-catechin] represented by the following formula 16; and
(2S)-4′,7-dihydroxyflavan-(4α->8)-catechin represented by the following formula 17 as an active ingredient:

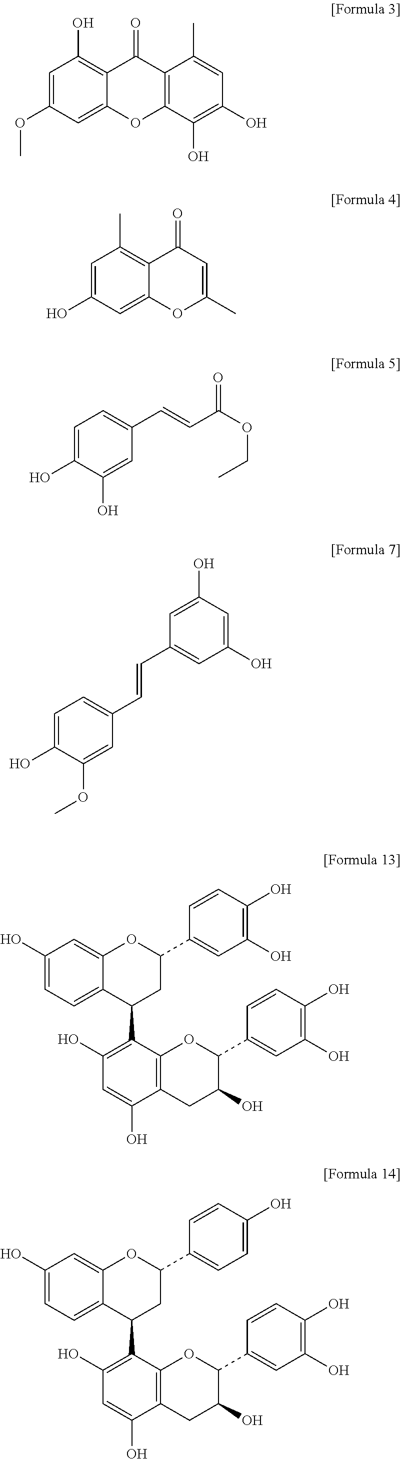 [SMILES: COc1cc(O)c2c(=O)c3c(C)cc(O)c(O)c3oc2c1 ; Cc1cc(=O)c2c(C)cc(O)cc2o1 ; CCOC(=O)/C=C/c1ccc(O)c(O)c1 ; COc1cc(/C=C/c2cc(O)cc(O)c2)ccc1O ; Oc1ccc2c(c1)O[C@H](c1ccc(O)c(O)c1)C[C@H]2c1c(O)cc(O)c2c1O[C@H](c1ccc(O)c(O)c1)[C@@H](O)C2 ; Oc1ccc([C@@H]2C[C@@H](c3c(O)cc(O)c4c3O[C@H](c3ccc(O)c(O)c3)[C@@H](O)C4)c3ccc(O)cc3O2)cc1]

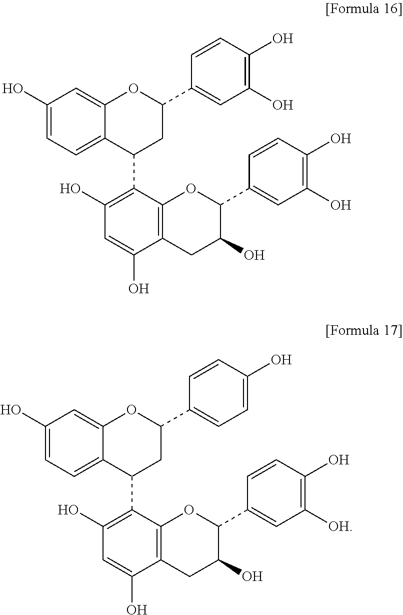 [SMILES: Oc1ccc2c(c1)O[C@H](c1ccc(O)c(O)c1)C[C@@H]2c1c(O)cc(O)c2c1O[C@H](c1ccc(O)c(O)c1)[C@@H](O)C2 ; Oc1ccc([C@@H]2C[C@H](c3c(O)cc(O)c4c3O[C@H](c3ccc(O)c(O)c3)[C@@H](O)C4)c3ccc(O)cc3O2)cc1]

### Advantageous Effects

In the present invention, the effect of Cassia mimosoides L. extract in treating allergic respiratory diseases was confirmed in an animal model of allergic asthma, and the anti-allergic effect of the compounds isolated from an ethanol extract of Cassia mimosoides L. was confirmed. Therefore, Cassia mimosoides L. extract of the present invention can be useful as a composition for preventing, improving, or treating allergic respiratory diseases, particularly allergic asthma or allergic bronchitis.

### DESCRIPTION OF FIGURES

FIG. 1 shows data confirming the inhibitory effect of ethanol extract of Cassia mimosoides L. on allergens in mast cell granules. In FIGS. 1 to 5, Con refers to the control group, IgE refers to the allergy-induced group, CNL20 refers to the group treated with 20 μg/ml of ethanol extract from Cassia mimosoides L., and CNL40 refers to the group treated with 40 μg/ml of ethanol extract from Cassia mimosoides L.

FIG. 2 is data confirming the inhibitory effect of ethanol extract of Cassia mimosoides L. on histamine production secreted by mast cells.

FIG. 3 shows data confirming the inhibitory effect of ethanol extract of Cassia mimosoides L. on TNF-α production secreted by mast cells.

FIG. 4 shows data confirming the inhibitory effect of ethanol extract of Cassia mimosoides L. on the production of interleukin-4 secreted by mast cells.

FIG. 5 shows data confirming the inhibitory effect of ethanol extract of Cassia mimosoides L. on the production of prostaglandin E2 secreted by mast cells.

FIG. 6 is data confirming the effect of ethanol extract of Cassia mimosoides L. on suppressing the number of inflammatory cells in bronchoalveolar lavage fluid. In the figures, Monocyte refers to a mononuclear cell, Lymphocyte refers to a lymphocyte cell, and PMN refers to an eosinophil cell. In addition, in FIGS. 6 to 11, Nor is the normal group, OVA is the allergic asthma-induced group, DEX is the dexamethasone-treated group (positive control), CN10 is the group treated with 10 mg/kg of Cassia mimosoides L. extract, and CN50 is the group treated with 50 mg/kg of Cassia mimosoides L. extract, and CN100 is the group treated with 100 mg/kg of Cassia mimosoides L. extract.

FIGS. 7A, 7B, and 7C show data confirming the effect of ethanol extract of Cassia mimosoides L. on suppressing the number of inflammatory cells in bronchoalveolar lavage fluid through flow cytometry. FIG. 7A is a diagram showing the absolute cell number, and FIGS. 7B and 7C are diagrams analyzing the cell number as percentage (%) of neutrophils and interstitial macrophages. In the figures, Neu refers to neutrophils, T refers to T cells, B refers to B cells, AM refers to alveolar macrophages, DC refers to dendritic cells, and IM refers to interstitial macrophages.

FIG. 8 is an image showing the effect of ethanol extract of Cassia mimosoides L. on suppressing immune cell infiltration through H&E staining.

FIG. 9A is an image and FIG. 9B is quantified data showing the effect of ethanol extract of Cassia mimosoides L. on suppressing immune cell infiltration through PAS (Periodic Acid-Schiff) staining.

FIG. 10 is data confirming the inhibitory effect of ethanol extract of Cassia mimosoides L. on serum IgE and ovalbumin-specific IgE concentrations.

FIG. 11 is analysis data for establishing yield-based optimal extraction and fractionation conditions from ethanol extract of Cassia mimosoides L.

FIG. 12 is a schematic diagram showing a method for separating compounds derived from ethanol extract of Cassia mimosoides L.

FIG. 13 shows data confirming the inhibitory effect of Cassia mimosoides L. extract on mast cell degranulation according to ethanol content.

FIG. 14 shows data confirming the inhibitory effect of Cassia mimosoides L. extract on mast cell degranulation depending on the type of fraction.

FIG. 15 is data confirming the inhibitory effect of mast cell degranulation inhibition of compounds derived from ethanol extract of Cassia mimosoides L.

FIG. 16 shows data confirming the inhibitory effect of compounds derived from ethanol extract of Cassia mimosoides L. on the production of histamine secreted by mast cells.

FIG. 17 shows data confirming the inhibitory effect of compounds derived from ethanol extract of Cassia mimosoides L. on TNF-α production secreted by mast cells.

FIG. 18 shows data confirming the inhibitory effect of compounds derived from ethanol extract of Cassia mimosoides L. on the production of interleukin-4 secreted by mast cells.

FIG. 19 shows data confirming the inhibitory effect of compounds derived from an ethanol extract of Cassia mimosoides L. on the production of prostaglandin E2 secreted by mast cells.

### MODE OF THE INVENTION

Hereinafter, the present invention will be described in detail.

In one aspect, the present invention relates to a pharmaceutical composition for the prevention or treatment of allergic respiratory diseases containing Cassia mimosoides L. extract as an active ingredient.

In addition, the present invention relates to a health functional food composition for preventing or improving respiratory allergic diseases containing Cassia mimosoides L. extract as an active ingredient.

In the present invention, Cassia mimosoides L. extract can be characterized as being extracted with ethanol, preferably 50% (v/v) to 100% (v/v) ethanol, more preferably 70% (v/v) to 95% (v/v) ethanol, as a solvent. Cassia mimosoides L. extract can be prepared using a conventional solvent according to a method known in the art, but solvents other than 50% (v/v) to 100% (v/v) ethanol may result in a lower content or no content of substances having anti-allergic properties in Cassia mimosoides L. extract.

In the present invention, the fraction can be characterized in that it is an ethyl acetate fraction of 60% (v/v) to 80% (v/v) ethanol extract of Cassia mimosoides L., preferably 70% (v/v) ethanol extract of Cassia mimosoides L.

In the present invention, the allergic respiratory disease can be characterized by allergic asthma, allergic bronchitis, or allergic rhinitis.

In the present invention, Cassia mimosoides L. extract can be characterized in that it inhibits the secretion of β-hexosaminidase, allergens present in the granules of mast cells, or inhibits the production of histamine, TNF-α, interleukin-4 (IL-4), or prostaglandin E2 secreted by mast cells.

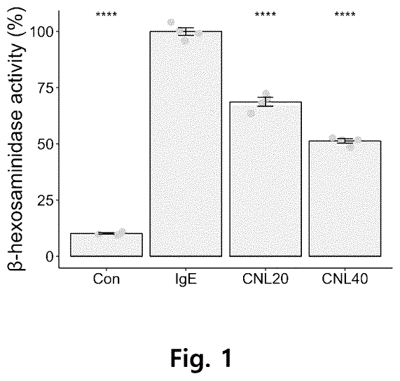

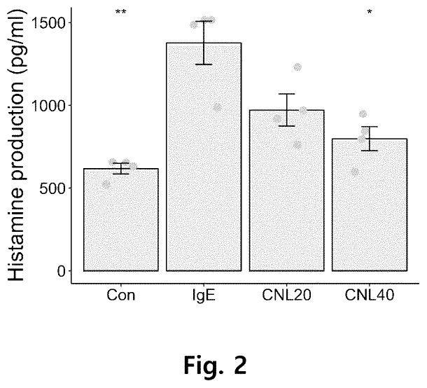

In a specific embodiment of the present invention, 70% ethanol extract of Cassia mimosoides L. was prepared, and its anti-allergy effect was confirmed. As a result, it was found that the secretion of allergens present in mast cell granules was inhibited by Cassia mimosoides L. ethanol extract (FIG. 1), and the production of histamine, TNF-α, interleukin-4, and prostaglandin E2 secreted by mast cells was inhibited (FIGS. 2 to 5).

In the present invention, Cassia mimosoides L. extract can inhibit or ameliorate allergic symptoms by reducing the number of eosinophils or inflammatory cells in the bronchi; reducing eosinophil infiltration; or reducing IgE in the serum.

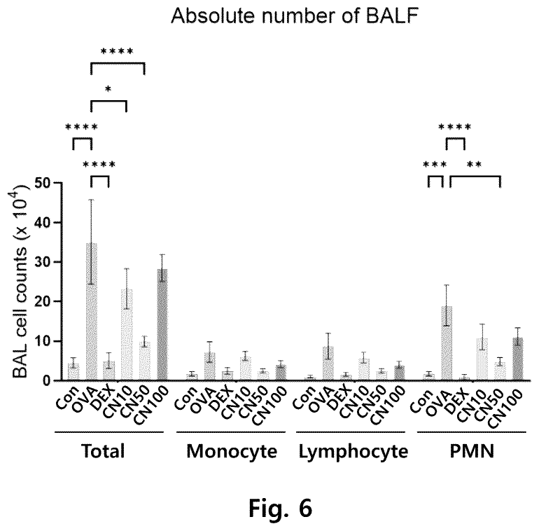

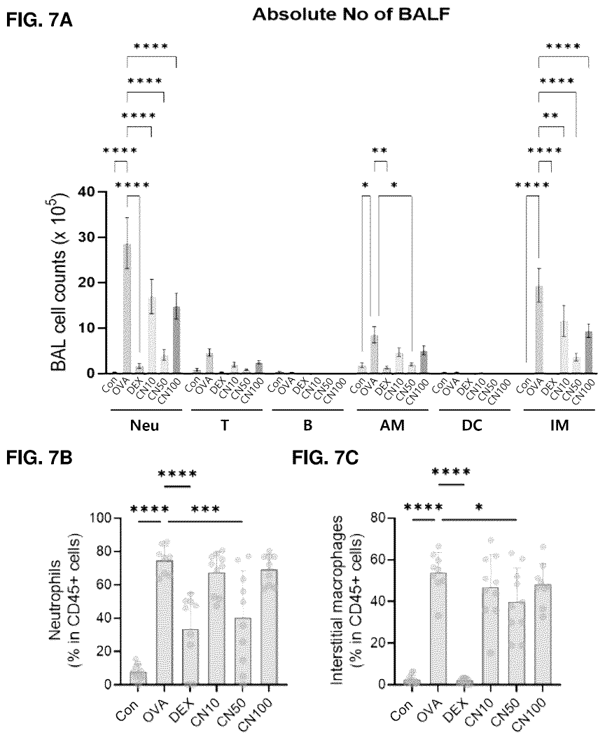

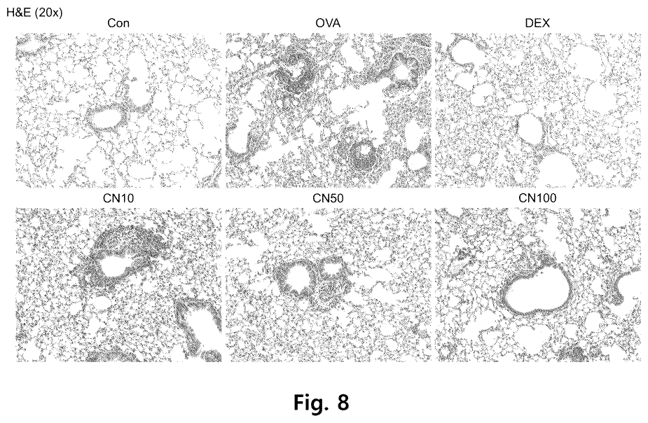

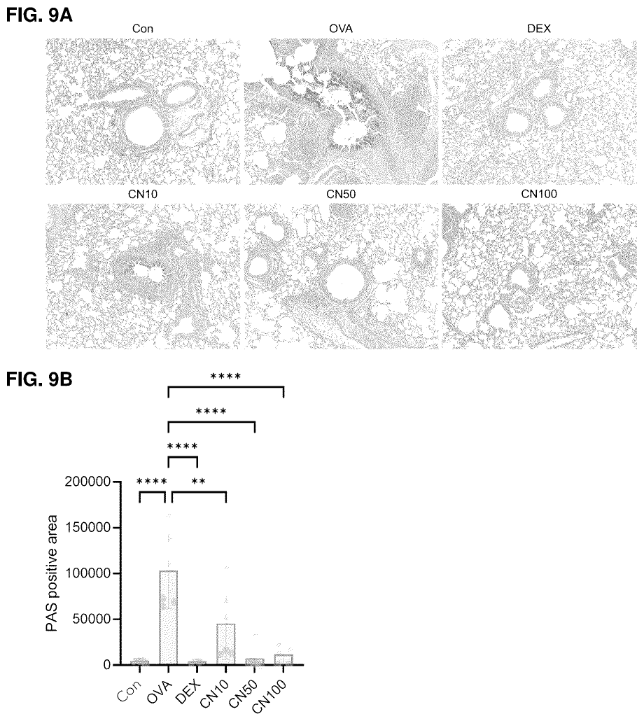

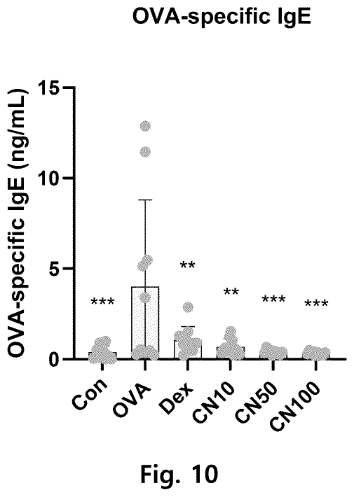

In another specific embodiment of the present invention, an animal model for inducing bronchial asthma, an allergic respiratory disease, was produced and treated with ethanol extract of Cassia mimosoides L., and as a result, it was found that the number of inflammatory cells in bronchoalveolar lavage fluid was reduced (FIGS. 6 and 7), the infiltration of eosinophils, an immune cell, was inhibited (FIGS. 8 and 9), and the concentration of IgE and ovalbumin-specific IgE in serum, which correlate with the severity of asthma, was inhibited (FIG. 10) in Cassia mimosoides L. ethanol extract-treated group.

In the present invention, Cassia mimosoides L. extract may contain any one or more compounds selected from the group consisting of:
Drimiopsin H represented by the following formula 3;
Altechromone A represented by the following formula 4;
Ethyl caffeate represented by the following formula 5;
Isorhapontigenin represented by the following formula 7;
KA-1 compound [(2S)-3′,4′,7-trihydroxyflavan-(4β->8)-catechin] represented by the following formula 13;
(2S)-4′,7-dihydroxyflavan-(4β->8)-catechin represented by the following formula 14;
KA-2 compound [(2S)-3′,4′,7-trihydroxyflavan-(4α->8)-catechin] represented by the following formula 16; and
(2S)-4′,7-dihydroxyflavan-(4α->8)-catechin represented by the following formula 17 as an active ingredient:

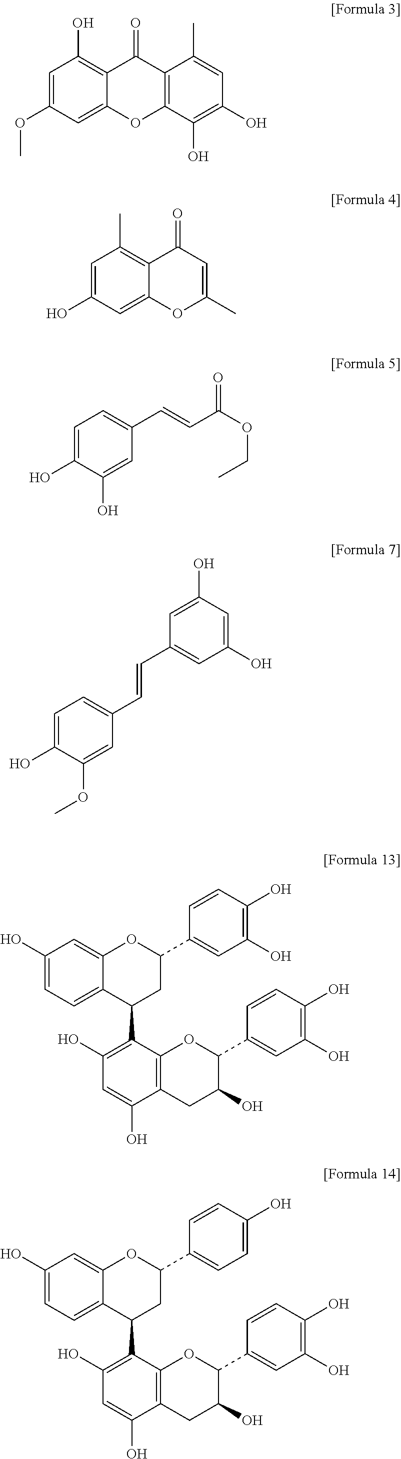 [SMILES: COc1cc(O)c2c(=O)c3c(C)cc(O)c(O)c3oc2c1 ; Cc1cc(=O)c2c(C)cc(O)cc2o1 ; CCOC(=O)/C=C/c1ccc(O)c(O)c1 ; COc1cc(/C=C/c2cc(O)cc(O)c2)ccc1O ; Oc1ccc2c(c1)O[C@H](c1ccc(O)c(O)c1)C[C@H]2c1c(O)cc(O)c2c1O[C@H](c1ccc(O)c(O)c1)[C@@H](O)C2 ; Oc1ccc([C@@H]2C[C@@H](c3c(O)cc(O)c4c3O[C@H](c3ccc(O)c(O)c3)[C@@H](O)C4)c3ccc(O)cc3O2)cc1]

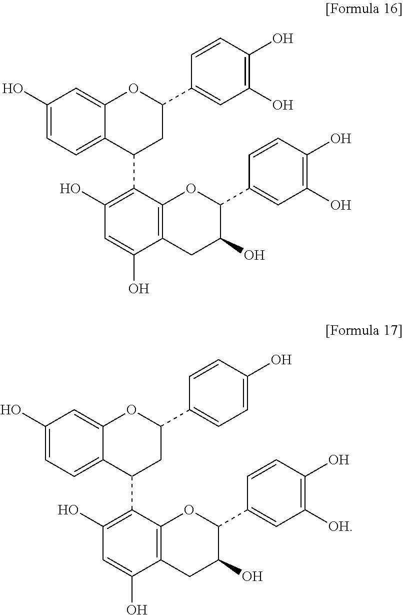 [SMILES: Oc1ccc2c(c1)O[C@H](c1ccc(O)c(O)c1)C[C@@H]2c1c(O)cc(O)c2c1O[C@H](c1ccc(O)c(O)c1)[C@@H](O)C2 ; Oc1ccc([C@@H]2C[C@H](c3c(O)cc(O)c4c3O[C@H](c3ccc(O)c(O)c3)[C@@H](O)C4)c3ccc(O)cc3O2)cc1]

Preferably, in the present invention, Cassia mimosoides L. can be characterized in that it comprises as active ingredients KA-1 compound represented by formula 13 and KA-2 compound represented by formula 16, which are specifically isolated from Cassia mimosoides L. ethanol extract.

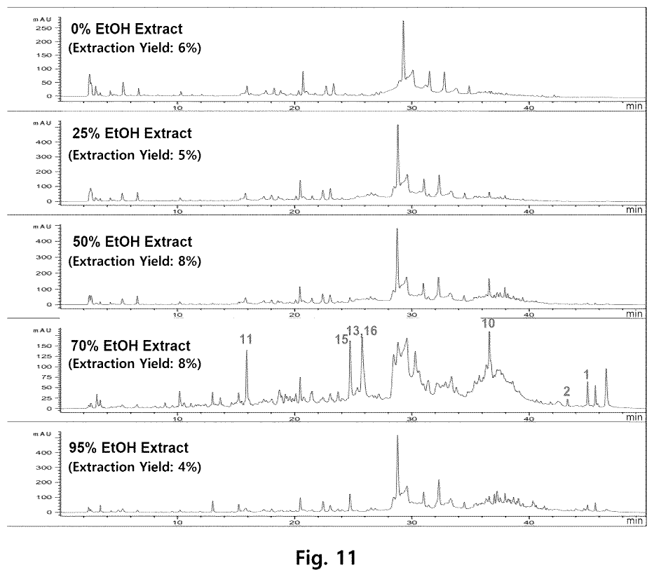

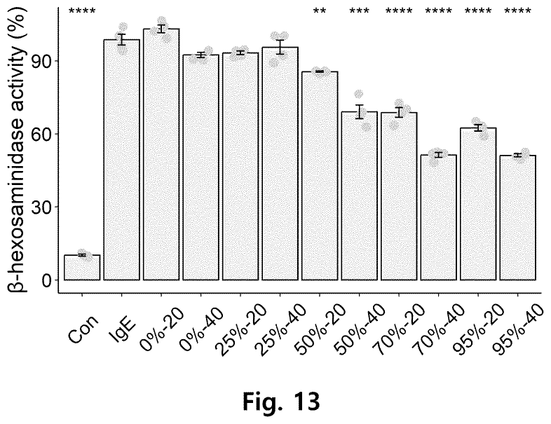

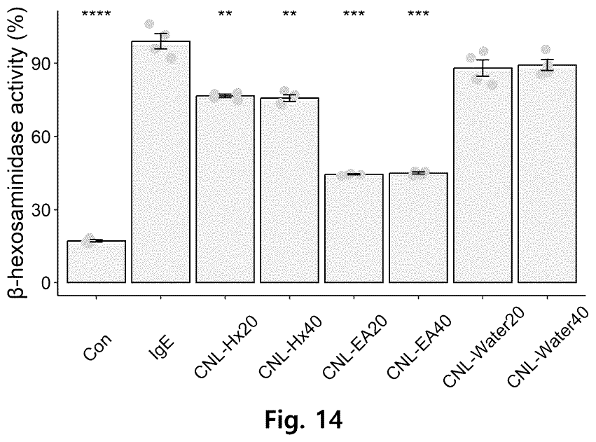

In another specific embodiment of the present invention, optimal extraction and fractionation conditions were established from 0%, 25%, 50%, 70%, or 95% ethanol extracts of Cassia mimosoides L., and it was found that 0%, 25%, 50%, and 95% ethanol extracts and 70% ethanol extract had different types of active ingredients that were isolated (FIG. 11). Furthermore, it was confirmed that 50%, 70%, and 95% ethanol extracts and ethyl acetate fractions of Cassia mimosoides L. effectively inhibited the secretion of allergens present in the granules of mast cells (FIGS. 13 and 14).

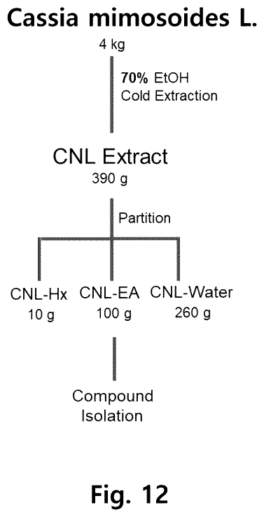

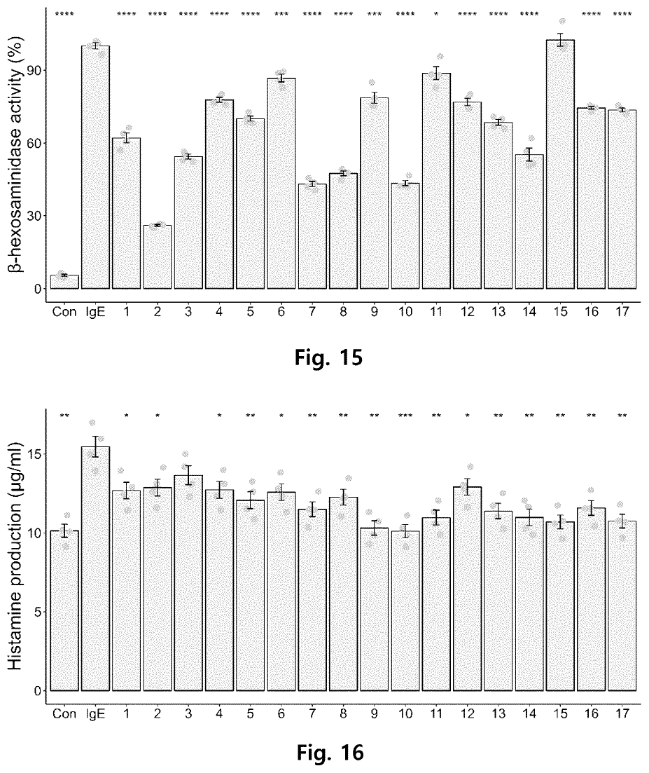

Furthermore, it was confirmed that 17 compounds isolated from ethyl acetate fraction of 70% ethanol extract by the method shown in the schematic of FIG. 12 inhibited degranulation of mast cells and inhibited the production of histamine, TNF-α, interleukin-4 and prostaglandin E2 (FIGS. 15 to 19). In particular, the compounds KA-1, represented by the formula 13, and KA-2, represented by the formula 16, which are specifically isolated from Cassia mimosoides L., showed excellent anti-allergic activity.

The pharmaceutical composition of the present invention can be formulated and used in various forms according to conventional methods. For example, it can be formulated into oral dosage forms such as powders, granules, tablets, capsules, suspensions, emulsions, syrups, etc., and can be formulated and used in the form of external preparations, suppositories, and sterile injection solutions. Depending on each dosage form, it may further include pharmaceutically acceptable carriers, excipients, and diluents. In addition, it can be formulated and used in the form of external preparations such as powders, granules, tablets, capsules, suspensions, emulsions, syrups, aerosols, etc., and sterile injection solutions according to conventional methods.

The carriers, excipients and diluents may include: lactose, dextrose, sucrose, oligosaccharides, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxy benzoate, propyl hydroxy benzoate, talc, magnesium stearate, mineral oil, and the like. When formulating the pharmaceutical composition, it can be prepared using diluents or excipients such as commonly used fillers, extenders, binders, wetting agents, disintegrants, and surfactants.

Solid preparations for oral administration may include tablets, pills, powders, granules, capsules, and the like, which are prepared by mixing the composition with at least one excipient, such as starch, calcium carbonate, sucrose, lactose, gelatin, and the like. In addition to simple excipients, lubricants such as magnesium stearate, talc can be also used. Liquid preparations for oral use may include suspensions, oral solutions, emulsions, and syrups. In addition to the commonly used simple diluents such as water and liquid paraffin, various excipients such as wetting agents, sweeteners, fragrances, and preservatives may be included. Preparations for parenteral administration may include sterilized aqueous solutions, non-aqueous solutions, suspensions, emulsions, freeze-dried preparations, suppositories, etc. Non-aqueous solvents and suspensions may include propylene glycol, polyethylene glycol, vegetable oil such as olive oil, and injectable ester such as ethyl oleate. As a base for suppositories, witepsol, macrogol, tween 61, cacao, laurin, glycerogelatin, etc. can be used.

As used herein, the term “administration” means providing the pharmaceutical composition of the present invention to an individual by any suitable method. The pharmaceutical compositions of the present invention can be administered in a therapeutically effective amount, which is an amount of the active ingredient or pharmaceutical composition that induces a biological or medical response in a tissue system, animal or human as conceived by the researcher, veterinarian, physician or other clinician, that is, an amount that induces relief of symptoms of the disease or disorder being treated. It will be apparent to those skilled in the art that the therapeutically effective dosage and number of doses of a pharmaceutical composition of the present invention will vary depending on the desired effect. Therefore, the optimal dosage to be administered can be readily determined by those skilled in the art and may be adjusted based on a variety of factors, including the type of disease, the severity of the disease, the content of the active ingredient and other ingredients in the composition, the type of formulation, the age, weight, general health status, gender and diet of the patient, the time of administration, the route of administration and the distribution ratio of the composition, the duration of treatment, and concomitant medications. The pharmaceutical compositions of the present invention can be administered to an individual by a variety of routes. For example, but not limited to, intravenous, intraperitoneal, intramuscular, intra-arterial, oral, intracardiac, intramedullary, intrathecal, transdermal, enteral, subcutaneous, sublingual, or topical administration. The pharmaceutical compositions of the present invention can be administered in an amount from 1 to 10,000 mg/kg/day, and can be administered once daily or in several divided doses.

The health functional food composition of the present invention can be used as a health functional food, food additive, or dietary supplement. When using the composition of the present invention as a food additive, it can be used appropriately according to conventional methods, such as adding it as is or mixing it with other foods or food ingredients.

Additionally, the mixing amount of the health functional food composition may be appropriately changed depending on the purpose of use (prevention, health control, or therapeutic treatment). As a specific example, when producing a food or beverage, the composition of the present invention can be added in an amount of 15% by weight or less, preferably 10% by weight or less, based on the raw materials. However, in the case of long-term consumption for health and hygiene purposes or for health control purposes, the composition may be added in an amount below the above range, and the active ingredient may be used in an amount above the above range, as there are no safety concerns.

There is no particular limitation on the type of food, but examples of food to which the composition of the present invention can be added include meat, sausages, bread, chocolate, candy, snacks, confectionery, pizza, ramen, other noodles, gum, and ice cream. It may include dairy products, various soups, beverages, tea, drinks, alcoholic beverages, vitamin complexes, etc., and include all health foods in the conventional sense.

When the health functional food composition of the present invention can be manufactured into a beverage, it may contain additional ingredients such as various flavoring agents or natural carbohydrates like ordinary beverages. The natural carbohydrates may include monosaccharides such as glucose and fructose; disaccharides such as maltose and sucrose; natural sweeteners such as dextrin and cyclodextrin; synthetic sweeteners such as saccharin and aspartame, etc. The natural carbohydrate can be included in an amount of 0.01 to 10% by weight, preferably 0.01 to 0.1% by weight, based on the total weight of the food composition of the present invention.

The health functional food composition of the present invention may include, but are not limited to, various nutrients, vitamins, electrolytes, flavoring agents, colorants, pectic acid and its salts, alginic acid and its salts, organic acids, protective colloidal thickeners, pH adjusters, stabilizers, preservatives, glycerin, alcohol, carbonation agents used in carbonated beverages, and the like, and may include pulp for the preparation of natural fruit juices, fruit juice drinks, and vegetable drinks. These ingredients may be used independently or in combination. The proportions of the above additives are not substantially limited, but are preferably included in the range of 0.01 to 0.1 wt % relative to the total weight of the food composition of the present invention.

Hereinafter, the present invention will be described in more detail through examples.

These examples are only for illustrating the present invention, and it will be apparent to those skilled in the art that the scope of the present invention is not to be construed as limited by these examples.

### Example 1

Preparation of Cassia mimosoides L. Extract

Cassia mimosoides L. was purchased at Gyeongdong Market. The extract was obtained by cold soxhlet extraction by mixing 4 kg of appropriately sized Cassia mimosoides L. and 24 liters of 70% ethanol in an extraction vessel, and then filtering through the extraction solvent filter paper to obtain the extract. The above extraction process was repeated three times, and the solvent was then concentrated under reduced pressure and dried to obtain 390 g of extract.

### Example 2

Inhibitory Effect of Cassia mimosoides L. Extract on Secretion of Allergens within Mast Cell Granules

In the present invention, it was confirmed whether Cassia mimosoides L. extract inhibited the secretion of allergens present in the granules of mast cells.

First, rat basophilic leukocyte (RBL-2H3, American Type Culture Collection, USA) mast cells were cultured in minimal medium containing antibiotics and 10% bovine serum. After culturing, the cells were collected with trypsin, placed in a 24-well microtiter plate at 2×10⁵ cells/well, and cultured until they grew to 80% confluence. The medium was replaced with PIPES buffer (25 mM PIPES, PH 7.2, 159 mM NaCl, 5 mM KCl, 0.4 mM MgCl₂, 1 mM CaCl₂), 5.6 mM glucose, and 0.1% BSA) for the cultured cells, and then ethanol extract of Cassia mimosoides L. prepared in <Example 1> was added to concentrations of 20 μg/ml and 40 μg/ml, respectively, and cultured for 1 hour. After 1 hour, DNP-BSA was added to a final concentration of 200 μg/ml, and stimulation was induced for 30 minutes.

The degree of secretion of allergy-inducing substances was determined by measuring the activity of beta-hexosaminidase, a marker of degranulation secreted in the medium, and the activity of beta-hexosaminidase was determined as the amount of p-nitrophenol liberated from p-nitrophenyl-acetyl-β-D-glucosaminide (Funaba M. et al., Cell Biol. Int., 27:879-85, 2003).

As a result, as shown in FIG. 1, it was confirmed that ethanol extract of Cassia mimosoides L. effectively suppressed the secretion of allergens present in the granules of mast cells in a concentration-dependent manner.

### Example 3

Inhibitory Effect of Cassia mimosoides L. Extract on Histamine Production Secreted by Mast Cells

In the present invention, it was confirmed whether Cassia mimosoides L. extract inhibited the production of histamine in mast cells.

First, RBL-2H3 mast cells were seeded at 2×10⁵ cells/well in a 24-well microtiter plate containing Dulbecco's Modified Eagle's Media (DMEM) medium containing 2.5% fetal bovine serum, and cultured until they grew to 80% confluence. Afterwards, ethanol extract of Cassia mimosoides L. prepared in <Example 1> was added to serum-free DMEM medium at a concentration of 20 μg/ml and 40 μg/ml, respectively, and mast cells were cultured for 24 hours. After treating mast cells with DNP-BSA for 30 minutes, the cell culture fluid was collected, centrifuged, and only the supernatant was harvested. The amount of histamine in the supernatant was confirmed using an EIA kit (Bertin Pharma, France).

As a result, as shown in FIG. 2, it was confirmed that ethanol extract of Cassia mimosoides L. effectively inhibited histamine production in mast cells in a concentration-dependent manner.

### Example 4

Inhibitory Effect of Cassia mimosoides L. Extract on TNF-α Production Secreted by Mast Cells

In the present invention, it was confirmed whether Cassia mimosoides L. extract inhibited the production of TNF-α in mast cells.

First, RBL-2H3 mast cells were seeded at 2×10⁵ cells/well in a 24-well microtiter plate containing Dulbecco's Modified Eagle's Media (DMEM) medium containing 2.5% fetal bovine serum, and cultured until they grew to 80% confluence. Afterwards, ethanol extract of Cassia mimosoides L. prepared in <Example 1> was added to serum-free DMEM medium at a concentration of 20 μg/ml and 40 μg/ml, respectively, and mast cells were cultured for 24 hours. After treating mast cells with DNP-BSA for 30 minutes, the cell culture fluid was collected, centrifuged, and only the supernatant was harvested. The amount of TNF-α in the supernatant was confirmed using an ELISA kit (Invitrogen, USA).

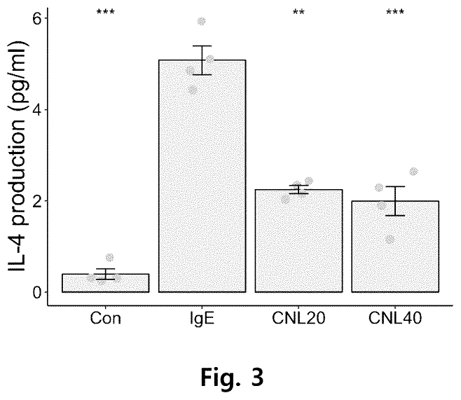

As a result, as shown in FIG. 3, it was confirmed that ethanol extract of Cassia mimosoides L. effectively inhibited TNF-α production in mast cells in a concentration-dependent manner.

### Example 5

Inhibitory Effect of Cassia mimosoides L. Extract on Interleukin-4 Production Secreted by Mast Cells

In the present invention, it was confirmed whether Cassia mimosoides L. extract inhibited the production of interleukin 4 (IL-4) in mast cells.

First, RBL-2H3 mast cells were seeded at 2×10⁵ cells/well in a 24-well microtiter plate containing Dulbecco's Modified Eagle's Media (DMEM) medium containing 2.5% fetal bovine serum, and cultured until they grew to 80% confluence. Afterwards, ethanol extract of Cassia mimosoides L. prepared in <Example 1> was added to serum-free DMEM medium at a concentration of 20 μg/ml and 40 μg/ml, respectively, and mast cells were cultured for 24 hours. After treating mast cells with DNP-BSA for 30 minutes, the cell culture fluid was collected, centrifuged, and only the supernatant was harvested. The amount of interleukin-4 in the supernatant was confirmed using an ELISA kit (Invitrogen, USA).

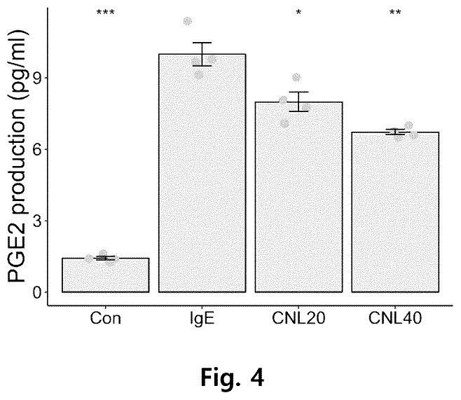

As a result, as shown in FIG. 4, it was confirmed that ethanol extract of Cassia mimosoides L. effectively inhibited the production of interleukin-4 in mast cells in a concentration-dependent manner.

### Example 6

Inhibitory Effect of Cassia mimosoides L. Extract on Prostaglandin E2 Production Secreted by Mast Cells

In the present invention, it was confirmed whether Cassia mimosoides L. extract inhibited the production of prostaglandin E2 (PGE2) in mast cells.

First, RBL-2H3 mast cells were seeded at 2×10⁵ cells/well in a 24-well microtiter plate containing Dulbecco's Modified Eagle's Media (DMEM) medium containing 2.5% fetal bovine serum, and cultured until they grew to 80% confluence. Afterwards, ethanol extract of Cassia mimosoides L. prepared in <Example 1> was added to serum-free DMEM medium at a concentration of 20 μg/ml and 40 μg/ml, respectively, and mast cells were cultured for 24 hours. After treating mast cells with DNP-BSA for 30 minutes, the cell culture fluid was collected, centrifuged, and only the supernatant was harvested. The amount of prostaglandin E2 in the supernatant was confirmed using an ELISA kit (R&D systems, USA).

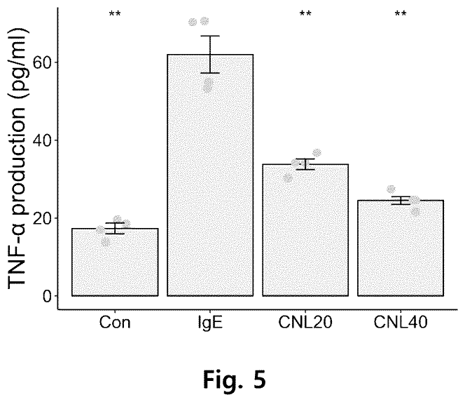

As a result, as shown in FIG. 5, it was confirmed that ethanol extract of Cassia mimosoides L. effectively inhibited prostaglandin E2 production in mast cells in a concentration-dependent manner.

### Example 7

Preparation of Animal Model for Inducing Allergic Bronchial Asthma and Administration of Cassia mimosoides L. Extract

In order to produce experimental animals for inducing allergic bronchial asthma, 6-week-old Balb/c female mice with an average weight of about 20 g were purchased from Orient Bio. The animals were acclimatized in a laboratory environment (25±1° C. temperature and 40±5% humidity) for 1 week, and animals with no abnormalities observed on basic physical examination were selected.

Mice were divided into six groups: normal (Control, PBS), allergic asthma-induced (OVA), dexamethasone (Sigma Aldrich), and three concentrations of Cassia mimosoides L. ethanol extract (10 mg/kg, 50 mg/kg, and 100 mg/kg). Dexamethasone, a steroid medication used to treat bronchial asthma, was used as a positive control.

Allergic bronchial asthma was induced by sensitization with ovalbumin (OVA; grade V; Sigma-Aldrich, USA) and aluminum hydroxide (Al(OH)₃, Sigma Aldrich), followed by inhalation of OVA using an ultrasonic nebulizer.

Specifically, mice in the six groups were injected intraperitoneally with 100 μg OVA and 1 mg Al(OH)₃ on days 0, 7, and 14, and then sensitized mice were inhaled with 5% OVA using an ultrasonic nebulizer for 20 minutes on days 24 and 27. For the dexamethasone group, dexamethasone was dissolved in a 0.5% (v/v) carboxymethylcellulose (CMC) solution and administered orally at 2.5 mg/kg on days 15 through 27. The three concentrations of Cassia mimosoides L. ethanol extract were administered orally at 10, 50, and 100 mg/kg of Cassia mimosoides L. ethanol extract dissolved in 0.5% (v/v) CMC solution in combination with OVA inhalation from day 15 to day 27, and mice were sacrificed on day 28.

### Example 8

Inhibitory Effect of Cassia mimosoides L. Extract on the Number of Inflammatory Cells in Bronchoalveolar Lavage Fluid

In order to confirm the anti-allergic asthma activity of the samples obtained in Example 7, an experiment was performed as follows by applying the method for evaluating inflammatory cells in bronchoalveolar lavage fluid described in the literature (Chang, Y. et al., Allergy Asthma Immunol Res, 14:99-116, 2022).

To obtain bronchoalveolar lavage fluid, an 18 G catheter was inserted into the bronchus and the bronchus was gently washed three times with 1 ml of sterile PBS containing 2% fetal bovine serum (FBS) to collect bronchoalveolar lavage fluid from the lungs. To confirm the number of inflammatory cells in the bronchoalveolar lavage fluid, each mouse bronchoalveolar lavage fluid collected was stained with trypan blue (T8154, Sigma) and the total number of cells, excluding dead cells, was counted using a hemocytometer (MIS-1401, Superior). After smearing the specimen using a centrifuge for cell collection (Cytospin, Shandon™), Diff-Quick staining (Sysmex, Switzerland) was performed to differentially count eosinophils and other inflammatory cells.

As a result, as shown in FIG. 6, the number of eosinophils and total inflammatory cells were significantly increased in the asthma-induced group (OVA) compared to the normal control group (Con), and the increased number of total inflammatory cells was significantly decreased in both the dexamethasone-treated group and Cassia mimosoides L. extract-treated group (CN, 10 mg/kg and 50 mg/kg), and the increased number of eosinophils was significantly decreased in both the dexamethasone-treated group and Cassia mimosoides L. extract-treated group (CN, 50 mg/kg).

### Example 9

Effect of Cassia mimosoides L. Extract on Suppressing the Number of Inflammatory Cells in Bronchoalveolar Lavage Fluid Through Flow Cytometry

Immunofluorescence staining was performed on the cells in the bronchoalveolar lavage fluid obtained in Example 8 at 4° C.

Anti-CD45-APC, anti-CD11b-FITC, and anti-Ly6G-PE were used to stain neutrophils, and anti-CD45-APC, anti-CD19-FITC, and anti-CD3-PE were used to stain T cells and B cells, anti-CD45-APC, anti-CD11c-FITC, anti-F4/80-PE were used to stain alveolar macrophages, interstitial macrophages, and dendritic cells. After reacting at 4° C. for 1 hour, the cells were washed three times with phosphate-buffered saline, and then inflammatory cell analysis was performed using flow cytometry (BD FACSVerse™ Cell Analyzer, BD Biosciences). Using the FlowJo program, neutrophils, T-cells, B-cells, alveolar macrophages, interstitial macrophages, and dendritic cells were analyzed in percentages (%) (FIG. 7B and FIG. 7C), which were then applied to the total cell count to produce an absolute cell count (FIG. 7A).

As a result, as shown in FIGS. 7A, 7B, and 7C, the cell counts of neutrophils, alveolar macrophages, and interstitial macrophages were significantly increased in the asthma-induced group (OVA) compared to the normal control group (Con), and each of the increased cell counts was significantly decreased in the dexamethasone-treated group and Cassia mimosoides L. extract-treated group (CN, 10 mg/kg, 50 mg/kg, 100 mg/kg).

In addition, neutrophils and interstitial macrophages were significantly increased in the OVA group compared to the normal control group (Con) in terms of cell counts obtained by flow cytometry, and the percentage of each increased cell was significantly decreased in the dexamethasone-treated group and Cassia mimosoides L. extract-treated group (CN, 50 mg/kg).

### Example 10

Inhibitory Effect of Cassia mimosoides L. Extract on Immune Cell Infiltration Through Histopathological Analysis

In order to confirm the infiltration of inflammatory cells, lungs were removed from each group of mice in Example 7, and then permanent tissue sections were prepared at 4 μm thickness through conventional formalin fixation and paraffin embedding, followed by fematoxylin and eosin (H&E) staining.

As a result, as shown in FIG. 8, unlike the normal group, eosinophils infiltrated through the blood vessels of the lungs in the allergic asthma induction group by OVA treatment and their number increased significantly, but the infiltration of eosinophils was significantly inhibited in the dexamethasone-treated group and Cassia mimosoides L. extract-treated group (CN, 10 mg/kg, 50 mg/kg, 100 mg/kg).

Additionally, Periodic Acid-Schiff (PAS) staining was also performed to assess hyperplasia of lung tissue goblet cells, which are responsible for mucus and secretion. As a result of observing germ cells stained with PAS staining with a light microscope along the pulmonary blood vessels, as shown in FIGS. 9A and 9B, the number of goblet cells was significantly increased in the allergic asthma induction group, while the infiltration of eosinophils was significantly inhibited in the dexamethasone-treated group and Cassia mimosoides L. extract-treated group (CN, 10 mg/kg, 50 mg/kg, 100 mg/kg).

### Example 11

Inhibitory Effect of Cassia mimosoides L. Extract on IgE and Ovalbumin-Specific IgE Concentration in Serum

Immunoenzyme reaction method was used in serum to measure IgE and ovalbumin-specific IgE (OVA-specific IgE), which are correlated with the severity of asthma.

Serum collected from each group in Example 7 was reacted in an ELISA kit (BioLegend) for measuring OVA-specific IgE concentration. After stopping the reaction with 2N sulfuric acid solution, the spectral absorbance was measured at 450 nm using an absorbance measuring device.

As a result, as shown in FIG. 10, the concentration of ovalbumin-specific IgE in the serum was significantly increased in the asthma induction group (OVA), while the concentration of ovalbumin-specific IgE antibodies was significantly decreased in the dexamethasone-treated group and Cassia mimosoides L. extract-treated group (CN, 10 mg/kg, 50 mg/kg, 100 mg/kg).

Therefore, it was concluded that Cassia mimosoides L. extract can be useful for the prevention or treatment of allergies and asthma by inhibiting the concentration of ovalbumin-specific IgE.

### Example 12

Isolation of Active Ingredients Derived from Cassia mimosoides L. Extract

In the present invention, in order to isolate an active ingredient with anti-allergy effect from Cassia mimosoides L. extract, yield-based optimal extraction and fractionation conditions were established from Cassia mimosoides L. extract.

First, 10 g of appropriately sized Cassia mimosoides L. was subjected to cold soxhlet extraction with a mixture of 0% (100% water), 25%, 50%, 70%, or 95% ethanol, respectively, and then filtered through extraction solvent filter paper to obtain an extract. The chemical components of Cassia mimosoides L. extract prepared above were analyzed using HPLC, LC-MS, and NMR analysis methods. The extract was loaded at 10 mg/ml and subjected to HPLC analysis. Table 1 below shows the HPLC analysis conditions, and Table 2 shows the compound names corresponding to each peak number in FIG. 11. Afterwards, the extract was subjected to systematic fractionation using hexane, ethyl acetate, and water.

As a result of comparing the extraction yield according to the solvent type, as shown in FIG. 11, the extraction yields of 50% ethanol extract and 70% ethanol extract were higher, and in particular, 0%, 25%, 50%, and 95% ethanol extracts and 70% ethanol extract had different types of isolated active ingredients.

According to the separation method shown in the schematic diagram of FIG. 12, 17 types of active ingredients were separated from the ethyl acetate extract of 70% ethanol extract of Cassia mimosoides L. (Table 2). Specifically, 4 kg of Cassia mimosoides L. were extracted three times with 70% ethanol, and then the solvent was concentrated and dried under reduced pressure to obtain 390 g of extract. Afterwards, the ethyl acetate layer was further separated using a silica gel open column under hexane-ethyl acetate gradient (hexane-EA gradient; 10:0 to 0:10) conditions to obtain five fractions. Among the five fractions, the third separated material was separated by molecular weight using a Sephadex open column under 50% methanol conditions, and 17 compounds were obtained using prep-MPLC.

TABLE 1
HPLC analysis conditions

| | Water | Acetonitrile |
|---|---|---|
| Time | (0.1% formic acid) | (0.1% formic acid) |
| 0 | 95 | 5 |
| 5 | 90 | 10 |
| 15 | 80 | 20 |
| 30 | 70 | 30 |
| 35 | 50 | 50 |
| 42 | 0 | 100 |
| 50 | 5 | 95 |

Column: Hydrosphere C18 (Ø4.6 × 250 mm, 5 μm)
Flow rate: 1.0 mL/min
Injection volume: 10 μl
Detection wavelength: 280 nm
Running time: 50 min
Column temperature: 30° C.

TABLE 2
17 compounds derived from Cassia mimosoides L. extract

| No. | Compound | |
|---|---|---|
| One | Chrysophanol | 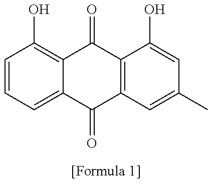 [SMILES: Cc1cc(O)c2c(c1)C(=O)c1cccc(O)c1C2=O] |
| 2 | Emodin | 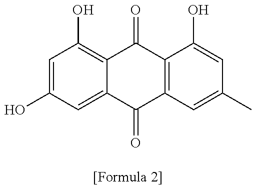 [SMILES: Cc1cc(O)c2c(c1)C(=O)c1cc(O)cc(O)c1C2=O] |
| 3 | Drimiopsin H | 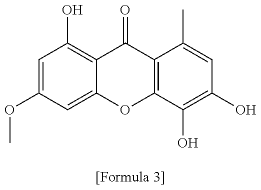 [SMILES: COc1cc(O)c2c(=O)c3c(C)cc(O)c(O)c3oc2c1] |
| 4 | Altechromone A | 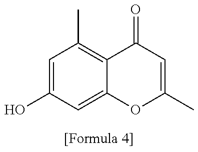 |
| 5 | Ethyl caffeate | 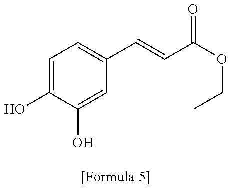 |
| 6 | p-coumaric acid | 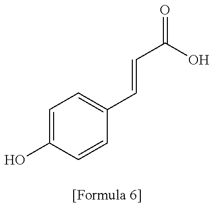 [SMILES: O=C(O)/C=C/c1ccc(O)cc1] |
| 7 | Isorhapontigenin | 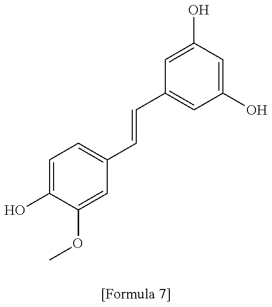 [SMILES: COc1cc(/C=C/c2cc(O)cc(O)c2)ccc1O] |
| 8 | Resveratrol | 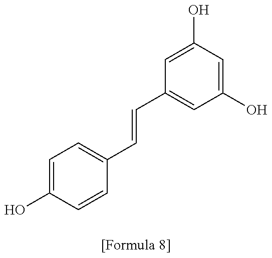 [SMILES: Oc1ccc(/C=C/c2cc(O)cc(O)c2)cc1] |
| 9 | Eriodictyol | 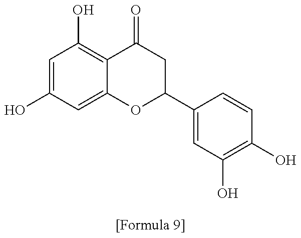 [SMILES: O=C1CC(c2ccc(O)c(O)c2)Oc2cc(O)cc(O)c21] |
| 10 | Luteolin | 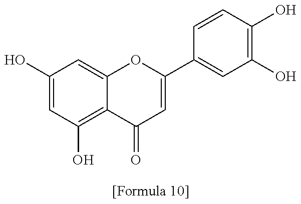 [SMILES: O=c1cc(-c2ccc(O)c(O)c2)oc2cc(O)cc(O)c12] |
| 11 | Catechin | 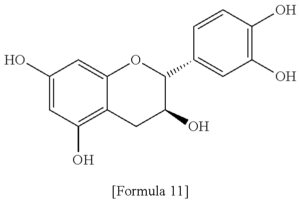 |
| 12 | Apigenin | 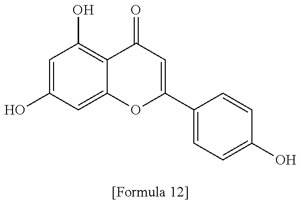 [SMILES: O=c1cc(-c2ccc(O)cc2)oc2cc(O)cc(O)c12] |
| 13 | KA-1 [(2S)-3′,4′,7-trihydroxyflavan-(4 β - >8)-catechin] | 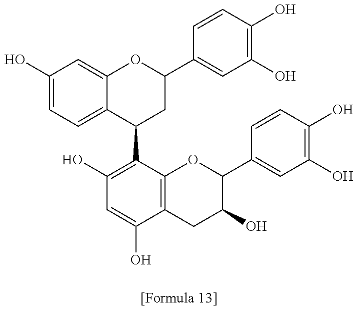 [SMILES: Oc1ccc2c(c1)OC(c1ccc(O)c(O)c1)C[C@H]2c1c(O)cc(O)c2c1OC(c1ccc(O)c(O)c1)[C@@H](O)C2] |
| 14 | (2S)-4′,7-dihydroxyflavan-(4 β ->8)- catechin | 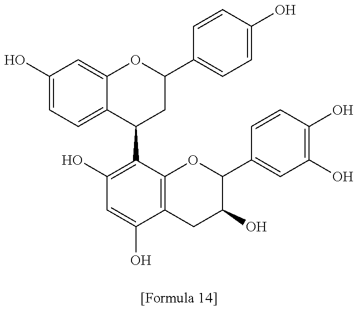 [SMILES: Oc1ccc(C2C[C@@H](c3c(O)cc(O)c4c3OC(c3ccc(O)c(O)c3)[C@@H](O)C4)c3ccc(O)cc3O2)cc1] |
| 15 | Isoquercitrin | 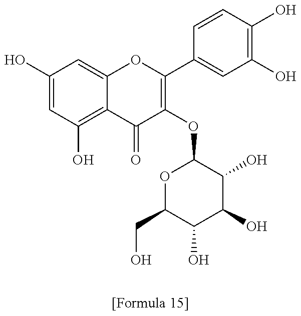 [SMILES: O=c1c(O[C@@H]2O[C@H](CO)[C@@H](O)[C@H](O)[C@H]2O)c(-c2ccc(O)c(O)c2)oc2cc(O)cc(O)c12] |
| 16 | KA-2 [(2S)-3′,4′,7-trihydroxyflavan-(4 α - >8)-catechin] | 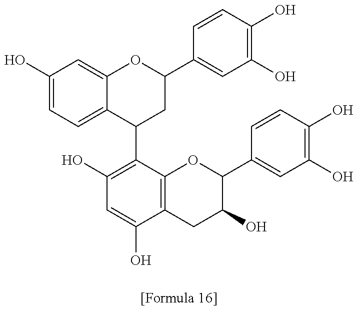 [SMILES: Oc1ccc2c(c1)OC(c1ccc(O)c(O)c1)CC2c1c(O)cc(O)c2c1OC(c1ccc(O)c(O)c1)[C@@H](O)C2] |
| 17 | (2S)-4′,7-dihydroxyflavan-(4 α ->8)- catechin | 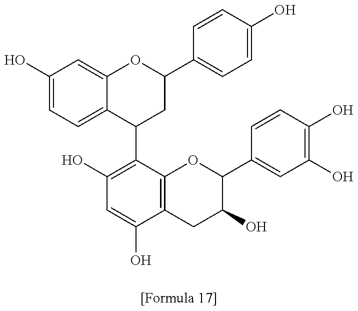 [SMILES: Oc1ccc(C2CC(c3c(O)cc(O)c4c3OC(c3ccc(O)c(O)c3)[C@@H](O)C4)c3ccc(O)cc3O2)cc1] |

### Example 13

Confirmation of Anti-Allergic Activity of Cassia mimosoides L. Extract by Solvent or Fraction

In the present invention, in order to actually confirm the anti-allergy effect of Cassia mimosoides L. extract according to ethanol content and type of fraction, mast cells were cultured in the same manner as in Example 2, and then the inhibition of mast cell degranulation by ethanol content and fraction type isolated in Example 12 above was confirmed.

First, rat basophilic leukocyte (RBL-2H3, American Type Culture Collection, USA) mast cells were cultured in minimal medium containing antibiotics and 10% bovine serum. After culturing, the cells were collected with trypsin, placed in a 24-well microtiter plate at 2×10⁵ cells/well, and cultured until 80% confluent. The medium was replaced with PIPES buffer (25 mM PIPES, pH 7.2, 159 mM NaCl, 5 mM KCl, 0.4 mM MgCl₂, 1 mM CaCl₂), 5.6 mM glucose, and 0.1% BSA) for the cultured cells, and then ethanol extracts (0, 25, 50, 70, 95% ethanol extracts) and fractions (ethyl acetate, hexane, and water) prepared in <Example 12> were added to reach concentrations of 20 μg/ml and 40 μg/ml, respectively, and incubated for 1 hour. After 1 hour, DNP-BSA was added to a final concentration of 200 μg/ml and stimulation was induced for 30 minutes.

The degree of allergen secretion was determined by measuring the activity of β-hexosaminidase, a marker of degranulation, which was secreted in the medium, and the activity of β-hexosaminidase was determined as the amount of p-nitrophenyl liberated from p-nitrophenyl-acetyl-β-D-glucosaminide (Funaba M. et al, Cell Biol. Int., 27:879-85, 2003).

As a result, as shown in FIGS. 13 and 14, it was confirmed that 50%, 70%, and 95% ethanol extracts and ethyl acetate fractions of Cassia mimosoides L. effectively inhibited the secretion of allergens present in the granules of mast cells, respectively.

### Example 14

Anti-Allergic Effect of Active Ingredients Derived from Cassia mimosoides L. Extract

### 14-1: Inhibitory Effect on Mast Cell Degranulation

Mast cells were cultured in the same manner as in Example 2, and then treated with 17 compounds isolated in Example 12 at a concentration of 20 μM, respectively.

As a result, as shown in FIG. 15, it was confirmed that mast cell degranulation was inhibited by 17 compounds derived from ethanol extract of Cassia mimosoides L., respectively.

### 14-2: Inhibitory Effect on Histamine Production

Mast cells were cultured in the same manner as in Example 3, and then treated with 17 compounds isolated in Example 12 at a concentration of 20 μM, respectively.

As a result, as shown in FIG. 16, it was confirmed that the production of histamine secreted by mast cells was suppressed by 17 compounds derived from ethanol extract of Cassia mimosoides L., respectively.

### 14-3: Inhibitory Effect on TNF-α Production

Mast cells were cultured in the same manner as in Example 4, and then treated with 17 compounds isolated in Example 12 at a concentration of 20 μM, respectively.

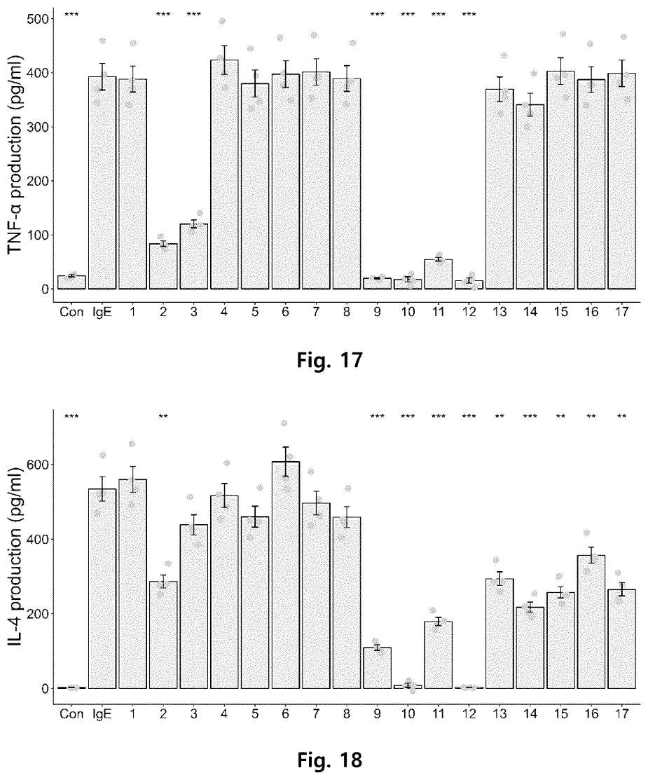

As a result, as shown in FIG. 17, it was confirmed that the production of TNF-α secreted by mast cells was suppressed by 17 compounds derived from ethanol extract of Cassia mimosoides L., respectively.

### 14-4: Inhibitory Effect on Interleukin-4 Production

Mast cells were cultured in the same manner as in Example 5, and then treated with 17 compounds isolated in Example 12 at a concentration of 20 μM, respectively.

As a result, as shown in FIG. 18, it was confirmed that the production of interleukin-4 secreted by mast cells was suppressed by 17 compounds derived from ethanol extract of Cassia mimosoides L., respectively.

### 14-5: Inhibitory Effect on Prostaglandin E2 Production

Mast cells were cultured in the same manner as in Example 6, and then treated with 17 compounds isolated in Example 12 at a concentration of 20 μM, respectively.

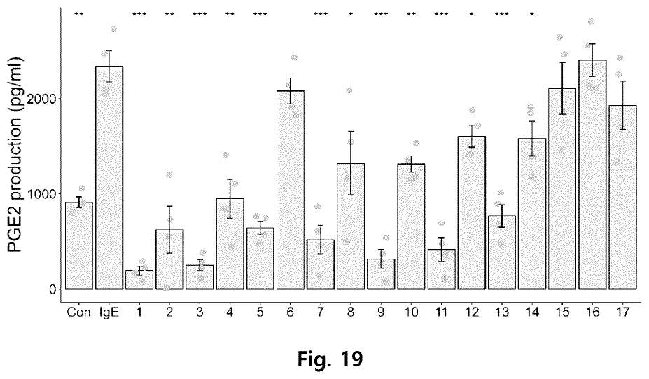

As a result, as shown in FIG. 19, it was confirmed that the production of prostaglandin E2 secreted by mast cells was inhibited by 17 compounds derived from ethanol extract of Cassia mimosoides L., respectively.

In particular, KA-1 compound represented by Formula 13 and KA-2 compound represented by Formula 16, which are specifically isolated only from Cassia mimosoides L., were found to have excellent efficacy in inhibiting mast cell degranulation and inhibiting the production of histamine, TNF-α, interleukin-4, and prostaglandin E2 secreted by mast cells.

1. A pharmaceutical composition for the prevention or treatment of allergic respiratory disease, comprising a Cassia mimosoides L. extract or fraction(s) thereof as an active ingredient.

2. The pharmaceutical composition for the prevention or treatment of allergic respiratory disease according to claim 1, wherein Cassia mimosoides L. extract is extracted using 50% (v/v) to 100% (v/v) ethanol as a solvent.

3. The pharmaceutical composition for the prevention or treatment of allergic respiratory disease according to claim 1, wherein the fraction is an ethyl acetate fraction of a 60% (v/v) to 80% (v/v) ethanol extract of Cassia mimosoides L.

4. The pharmaceutical composition for the prevention or treatment of allergic respiratory disease according to claim 1, wherein the allergic respiratory disease is allergic asthma, allergic bronchitis, or allergic rhinitis.

5. The pharmaceutical composition for the prevention or treatment of allergic respiratory disease according to claim 1, wherein Cassia mimosoides L. extract inhibits the secretion of beta-hexosaminidase, allergens present in the granules of mast cells, or inhibits the production of histamine, TNF-α, interleukin-4 (IL-4) or prostaglandin E2 secreted by mast cells.

6. The pharmaceutical composition for the prevention or treatment of allergic respiratory disease according to claim 1, wherein Cassia mimosoides L. extract reduces the number of eosinophils or inflammatory cells in the bronchial tubes; decreases eosinophil infiltration; or suppresses or improves allergic symptoms by reducing IgE in serum.

7. The pharmaceutical composition for the prevention or treatment of allergic respiratory disease according to claim 1, wherein Cassia mimosoides L. extract contains any one or more compounds selected from the group consisting of:
Drimiopsin H represented by the following formula 3;
Altechromone A represented by the following formula 4;
Ethyl caffeate represented by the following formula 5;
Isorhapontigenin represented by the following formula 7;
KA-1 compound [(2S)-3′,4′,7-trihydroxyflavan-(4β->8)-catechin] represented by the following formula 13;
(2S)-4′,7-dihydroxyflavan-(4β->8)-catechin represented by the following formula 14;
KA-2 compound [(2S)-3′,4′,7-trihydroxyflavan-(4α->8)-catechin] represented by the following formula 16; and
(2S)-4′,7-dihydroxyflavan-(4α->8)-catechin represented by the following formula 17 as an active ingredient:

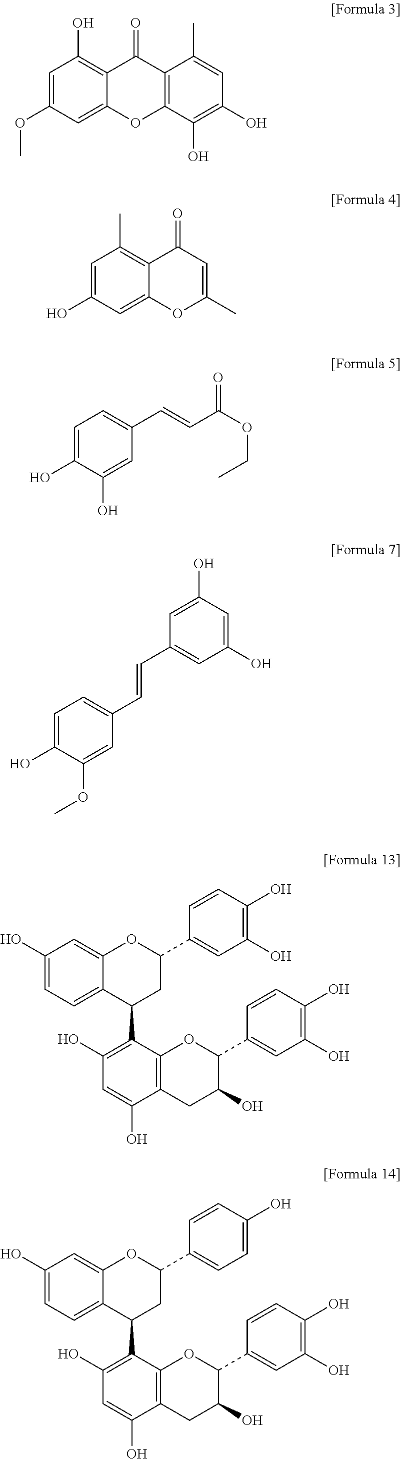 [SMILES: COc1cc(O)c2c(=O)c3c(C)cc(O)c(O)c3oc2c1 ; Cc1cc(=O)c2c(C)cc(O)cc2o1 ; CCOC(=O)/C=C/c1ccc(O)c(O)c1 ; COc1cc(/C=C/c2cc(O)cc(O)c2)ccc1O ; Oc1ccc2c(c1)O[C@H](c1ccc(O)c(O)c1)C[C@H]2c1c(O)cc(O)c2c1O[C@H](c1ccc(O)c(O)c1)[C@@H](O)C2 ; Oc1ccc([C@@H]2C[C@@H](c3c(O)cc(O)c4c3O[C@H](c3ccc(O)c(O)c3)[C@@H](O)C4)c3ccc(O)cc3O2)cc1]

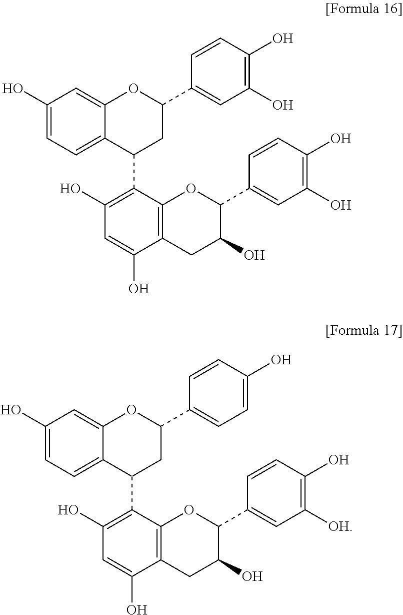 [SMILES: Oc1ccc2c(c1)O[C@H](c1ccc(O)c(O)c1)C[C@@H]2c1c(O)cc(O)c2c1O[C@H](c1ccc(O)c(O)c1)[C@@H](O)C2 ; Oc1ccc([C@@H]2C[C@H](c3c(O)cc(O)c4c3O[C@H](c3ccc(O)c(O)c3)[C@@H](O)C4)c3ccc(O)cc3O2)cc1]

8. A health functional food composition for preventing or improving allergic respiratory disease, comprising Cassia mimosoides L. extract as an active ingredient.

9. The health functional food composition for preventing or improving allergic respiratory disease according to claim 8, wherein Cassia mimosoides L. extract is extracted using 50% (v/v) to 100% (v/v) ethanol as a solvent.

10. The health functional food composition for preventing or improving allergic respiratory disease according to claim 8, wherein the fraction is an ethyl acetate fraction of a 60% (v/v) to 80% (v/v) ethanol extract of Cassia mimosoides L.

11. The health functional food composition for preventing or improving allergic respiratory disease according to claim 8, wherein the allergic disease is allergic asthma or allergic bronchitis.

12. The health functional food composition for preventing or improving allergic respiratory disease according to claim 8, wherein Cassia mimosoides L. extract inhibits the secretion of beta-hexosaminidase, allergens present in the granules of mast cells, or inhibits the production of histamine, TNF-α, interleukin-4 (IL-4) or prostaglandin E2 secreted by mast cells.

13. The health functional food composition for preventing or improving allergic respiratory disease according to claim 8, wherein Cassia mimosoides L. extract reduces the number of eosinophils or inflammatory cells in the bronchial tubes; decreases eosinophil infiltration; or suppresses or improves allergic symptoms by reducing IgE in serum.

14. The health functional food composition for preventing or improving allergic respiratory disease according to claim 8, wherein Cassia mimosoides L. extract contains any one or more compounds selected from the group consisting of:
Drimiopsin H represented by the following formula 3;
Altechromone A represented by the following formula 4;
Ethyl caffeate represented by the following formula 5;
Isorhapontigenin represented by the following formula 7;
KA-1 compound [(2S)-3′,4′,7-trihydroxyflavan-(4β->8)-catechin] represented by the following formula 13;
(2S)-4′,7-dihydroxyflavan-(4->8)-catechin represented by the following formula 14;
KA-2 compound [(2S)-3′,4′,7-trihydroxyflavan-(4α->8)-catechin] represented by the following formula 16; and
(2S)-4′,7-dihydroxyflavan-(4α->8)-catechin represented by the following formula 17 as an active ingredient: 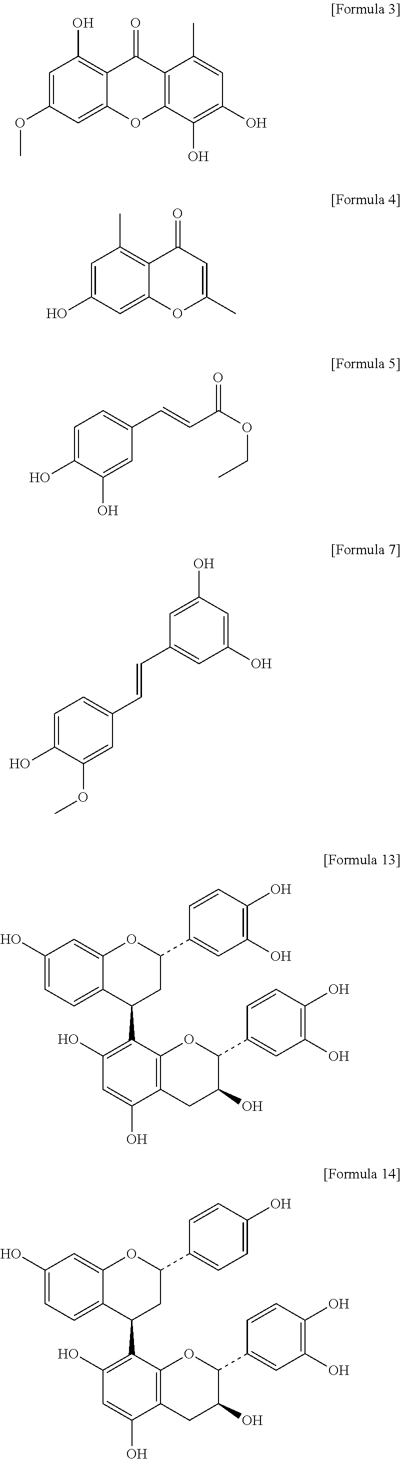 [SMILES: COc1cc(O)c2c(=O)c3c(C)cc(O)c(O)c3oc2c1 ; Cc1cc(=O)c2c(C)cc(O)cc2o1 ; CCOC(=O)/C=C/c1ccc(O)c(O)c1 ; COc1cc(/C=C/c2cc(O)cc(O)c2)ccc1O ; Oc1ccc2c(c1)O[C@H](c1ccc(O)c(O)c1)C[C@H]2c1c(O)cc(O)c2c1O[C@H](c1ccc(O)c(O)c1)[C@@H](O)C2 ; Oc1ccc([C@@H]2C[C@@H](c3c(O)cc(O)c4c3O[C@H](c3ccc(O)c(O)c3)[C@@H](O)C4)c3ccc(O)cc3O2)cc1] 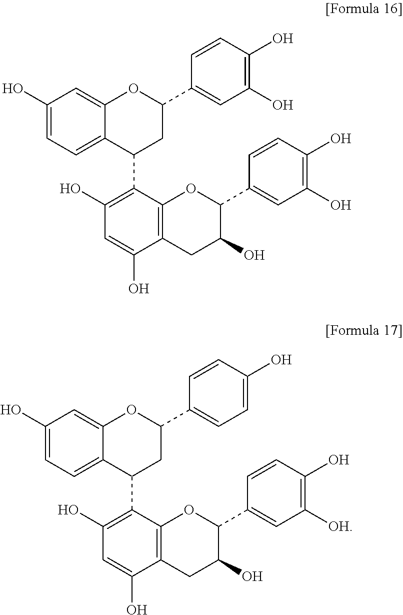 [SMILES: Oc1ccc2c(c1)O[C@H](c1ccc(O)c(O)c1)C[C@@H]2c1c(O)cc(O)c2c1O[C@H](c1ccc(O)c(O)c1)[C@@H](O)C2 ; Oc1ccc([C@@H]2C[C@H](c3c(O)cc(O)c4c3O[C@H](c3ccc(O)c(O)c3)[C@@H](O)C4)c3ccc(O)cc3O2)cc1]
